(19) United States
(12) Patent Application Publication
(10) Pub. No.: US 2022/0093230 A1
(43) Pub. Date: Mar. 24, 2022

(54) MOBILE HEALTH AND FITNESS REACTIVE IMMERSIVE ACTIVITY SPACE

(71) Applicant: ASEEM GUPTA, DUBAI (AE)

(72) Inventor: ASEEM GUPTA, DUBAI (AE)

(21) Appl. No.: 17/464,696

(22) Filed: Sep. 2, 2021

Related U.S. Application Data
(60) Provisional application No. 63/080,806, filed on Sep. 21, 2020.

(51) Int. Cl. G16H 20/30; G16H 40/67; G16H 70/20; A63B 71/06; A63B 24/00

(52) U.S. Cl. CPC G16H 20/30; A63B 2225/20; G16H 70/20; A63B 71/0622; A63B 24/0062; A63B 24/0084; A63B 2225/12; A63B 2071/0638; A63B 2225/74; A63B 2220/56; A63B 2220/833; A63B 2225/50; A63B 2071/0655; A63B 2071/0625; A63B 2225/11; A63B 2208/0233; A63B 2208/0252; G16H 40/67

(57) ABSTRACT

This invention relates to a mobile health and fitness reactive immersive activity space embodied with a plurality of modules and more particularly to a motorized or towed vehicle or vessel with its structural space embodied in modular assembly with sensory flooring and walls, providing a immersive platform for performing health assessments and treatments as well as physical and mental health fitness activities for functional training and therapy, using a plurality of sensors, electronics, electrical and ambient audio-visual stimulation that can be programmed to suit the specific needs of an individual or a group of individuals.

This is the complete specification of provisional filed U.S. patent application Ser. No. 63080806 (under 35 USC 111(b)), which claims priority to U.S. provisional Patent Application 63080806, filed 21 Sep. 2020. The present application has the same inventor as the aforementioned application.

### FIELD OF INVENTION

This invention relates to a mobile health and fitness reactive immersive activity space embodied with a plurality of modules and more particularly to a motorized or towed vehicle with its structural space embodied in modular assembly with sensory flooring and walls, providing a immersive platform for performing health assessments and treatments as well as physical and mental health fitness activities for functional training and therapy, using a plurality of sensors, electronics, electrical and ambient audio-visual stimulation that can be programmed to suit the specific needs of an individual or a group of individuals

### BACKGROUND OF THE INVENTION

COVID-19 has highlighted those inequalities in health and wellbeing that have made people more vulnerable to this disease compounded by the fact that a majority of the world population is overweight or obese and several million people die every year from causes related to Diabetes, Obesity and Heart Disease. The situation is reversible with timely health assessment and incremented physical activity.

The need for physical and mental fitness exercises is a priority from a human health and safety standpoint. Traditionally, fitness venues such as brick-and-mortar gyms, have focused on maximizing usage of real estate space by tightly packing users in physical fitness machine populated environments. Likewise, hospitals and medical establishments have focused on curing the problem rather than preventing them, leading to dramatic increase in the incidence of preventable lifestyle diseases which has reached epidemic proportions. Over the years, various systems and methodologies for promoting wellness, physical activity/education and fitness have been created. In the aftermath of the novel Corona Virus (COVID-19) pandemic, unforeseen new challenges have emerged in physical social space interaction which affects physical fitness activities in venues such as gyms, fitness studio's and every dedicated or shared workout spaces located indoors or outdoors where people participate in individual or group fitness activities. Prior disclosures have revealed mobile fitness units that transport fitness machines such as treadmills, rowers, exercise bikes and weight training equipment which, albeit their mobility, do not provide an alternative functional fitness system and are limited to physical fitness for gym going individuals only, without providing health assessments and treatments to cater to a variety of individual physical and mental health and fitness needs of people suffering from lifestyle diseases.

There is the intrinsic need for individuals to develop a strong immune system and to maintain a good physical and mental fitness level as a defense mechanism against sedentary lifestyle related muscle atrophy, obesity, diabetes 2 and cardio-vascular disease. Therefore, on one hand, while traditional means of physical fitness at fitness venues has been severely disabled during the COVID-19 lockdown, the demand for physical fitness has become one of the highest priorities for people of all ages in general.

Hence, there is the need for a solution that secures users health and safety concerns while still providing the user with guided individual and group exercises.

Thus, the present invention addresses specifically health assessment, treatment and physical activity considerations, by providing a novel mobile vehicle as a solution conveniently available to users at their own convenience of time and location, with an advantageous embodiment for a mobile reactive functional fitness immersive exercise platform involving eye, hand and body reaction-based fitness training for people of all ages and fitness levels.

The invention's novelty is that it provides a mobile health and fitness interactive and immersive activity space that delivers bespoke functional training and therapy as an efficient alternative to traditional brick-and-mortar gyms and mobile fitness units that are laden with machines and weight training equipment.

The invention provides a modular mobile interactive and immersive health and fitness space using reactive floor and walls for high intensity functional training, with the objective of reducing the fitness activity time by as much as eighty percent, while providing similar calorie burn as compared to a traditional cardio machines and weights driven exercise training system

### SUMMARY OF THE INVENTION

A mobile health and fitness reactive immersive activity space comprises a mobile physical space (100, 150, 170) capable of being transported for mobility and abbreviated with a reactive flexible wall and floor surface module (200). A mirror embedded leader board and streaming video instruction display module (300) a health assessment module (410). A retractable treatment and therapy couch module (420) and an immersive video exergaming module (500) provides high intensity and game based exercise content, based on user selection of the program list from the smartphone/tablet app (780), in conjunction with the libraries stored in the connected cloud (790) that, in one instance uses a projection display, via the projector (520) and the tactile projection screen surface (510) and response and scoring data is processed via the immersive spatial sensor (550) and made visible on the screen via the interactive immersive content (530).

Plurality of interactive sensory units including, but are not limited to, sensors (not shown) are capable of reading motion, pressure, weight, height, body measurements and bio-metric measurement that provide heart rate, blood pressure, blood oxygen level, electro-cardiography, electro-myography, lung capacity, blood glucose levels, to name a few, listed here for explanatory purposes only and without limitation to additional health sensors that are connected and integrated to the health sensor data aggregator (740).

The reactive wall and floor functional activity unit (200) with an addressable array of interactive light emitting diodes (220) and pressure sensors (225) encased inside a translucent top surface (230) and a rigid protective layer of flexible material (215) and a bottom layer of soft impact absorbing material (210).

A main controller (705) is embodied with a redundant high performance CPU and communication sub-systems equipped with wireless connectivity (706) which is equipped, but not limited to, using wired and wireless communication for interaction with sensors and equipment embodied with the mobile activity space (100, 150, 170), as well as connecting smartphones/tablet and smartwatch devices (780, 770) and a secure cloud platform (790)

### BRIEF DESCRIPTION OF THE DRAWINGS

It is to be noted, however, that the appended drawings illustrate only typical embodiments of this invention and are therefore not to be considered for limiting of its scope, for the invention may admit to other equally effective embodiments.

FIG. 1—reveals the physical form and attributes of the invention as embodied in a custom box truck/van chassis.

FIG. 2—a sectional view showing the modular elements embodies within the box that provide an integrated functional system for health assessment and treatment module, functional exercise using suspension and reaction training module, mirror embedded leaderboard and streaming video instruction display module and an immersive video exergaming module, embodied within an autonomous cloud and smart device connected customized box truck/van.

FIG. 3—reveals a sectional view of the internal layout of the truck/van box body showing a representative embodiment of a plurality of square tiles set side by side which together constitutes the reactive exercise physical surface on the wall and floor. The top of the reactive surface is represented with markings and designs to aid user during training.

FIG. 4—reveals the generic elements of the reactive surface embodied with pressure sensors and arrays of LED lights that provide visual cues to aid users during the training.

FIG. 5—reveals a sectional view of the various layered elements that are sandwiched together as a single bonded reactive element with visual cues from interactive LED lighting and pressure sensors to record and respond to tactile impact from hands and feet.

FIG. 6—reveals the mirror embedded leaderboard and streaming video instruction display on the back wall of the body.

FIG. 7—reveals the perspective view showing a large screen display positioned behind a two-way acrylic mirror which aids users during exercise as well as fulfills a video display function as and when required to show training videos and leader board information.

FIG. 8—reveals the wall and floor mounted health assessment platform embodied with a plurality of body sensors for reading and measuring.

FIG. 9—demonstrates user interaction with the health assessment platform.

FIG. 10—represents the folded aspect.

FIG. 11—reveals a ninety-degree unfolded seated aspect.

FIG. 12—reveals a one-hundred-eighty-degree flatbed aspect.

FIG. 13—demonstrates a patient and professional interaction on the couch.

FIG. 14—reveals the ceiling mounted spatial sensing projector and projection wall surface.

FIG. 15—demonstrates a user exercise with the user interacting with the projected content on the wall surface and the gaming aspect of score display.

FIG. 16—demonstrates a plurality of interactive isometric high intensity exercise with the wall and floor reactive training module and another aspect of yoga training and quiet meditation.

FIG. 17—demonstrates users in a plurality of suspension and resistance-based body weight training aspect of the reactive immersive activity space.

FIG. 18—reveals the transportable and un-expanded tow trailer aspect to demonstrate the transportability of the integrated system.

FIG. 19—reveals the parked and expanded aspect of the tow-trailer embodiment with access door and steps.

FIG. 20—reveals a representative layout of the previously revealed modules as assembled in a expanded space as compared to the compact box truck/van.

FIG. 21—reveals the side view of a watercraft showing a lower enclosed deck and a flybridge customized to function as a reactive immersive floating activity space.

FIG. 22—reveals a representative layout of the previously revealed modules as assembled inside the main cabin of the watercraft.

FIG. 23—reveals a technical hierarchy of the physical and application aspects of the system layers presented logically with text and header references that define the system components where numbered.

FIG. 24 provides system data and command flows between the main controller, the smartphone / tablet/smartwatch app and the cloud platform that make up a functional system for individual and group interactions.

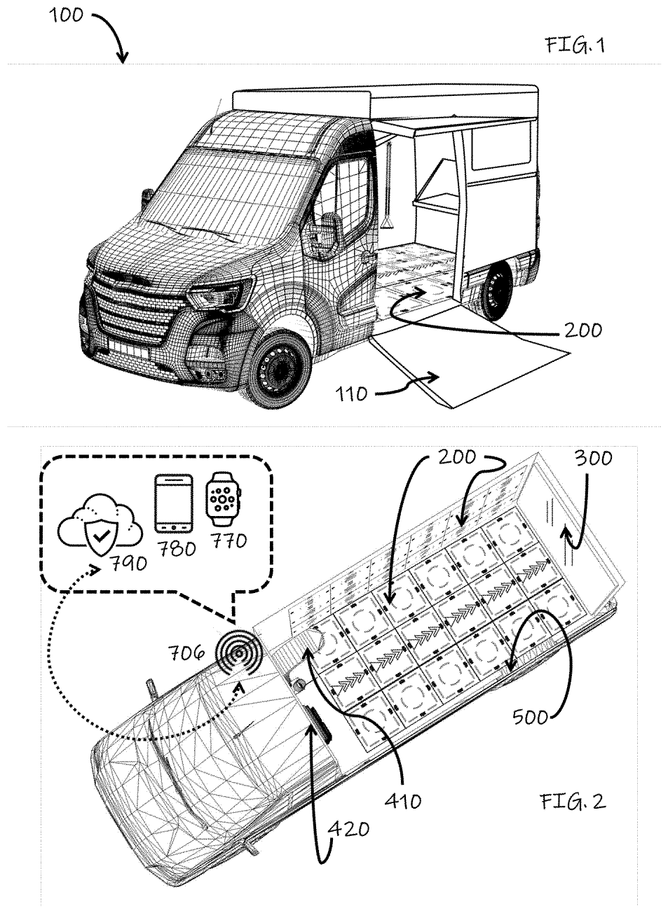

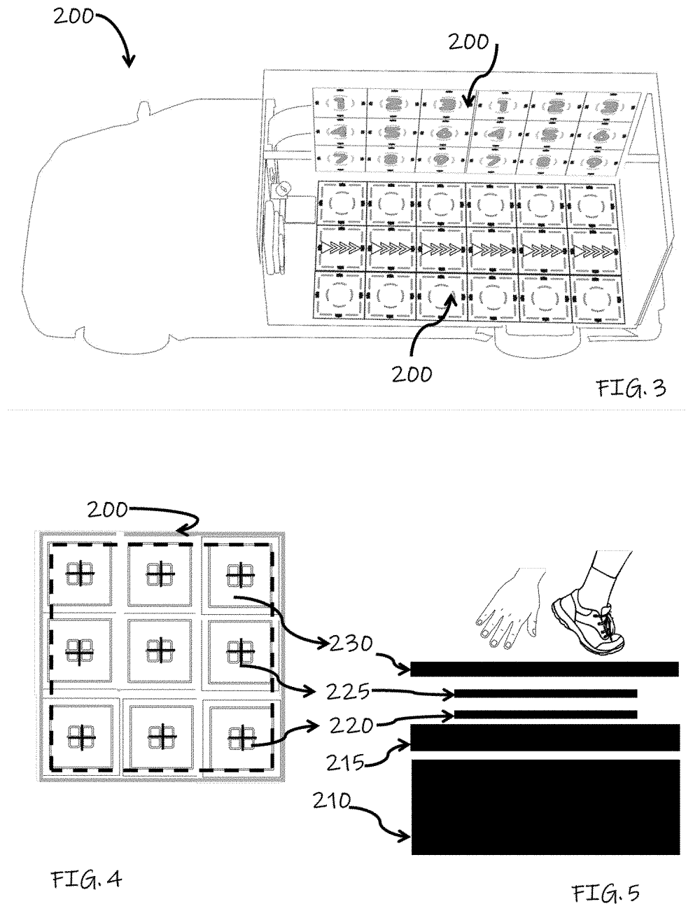

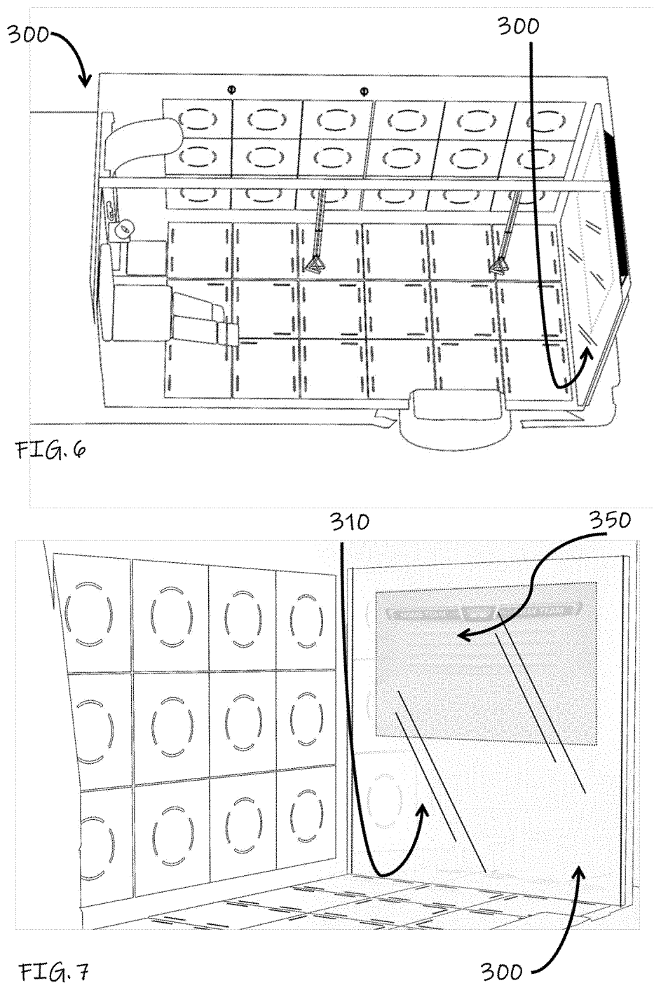

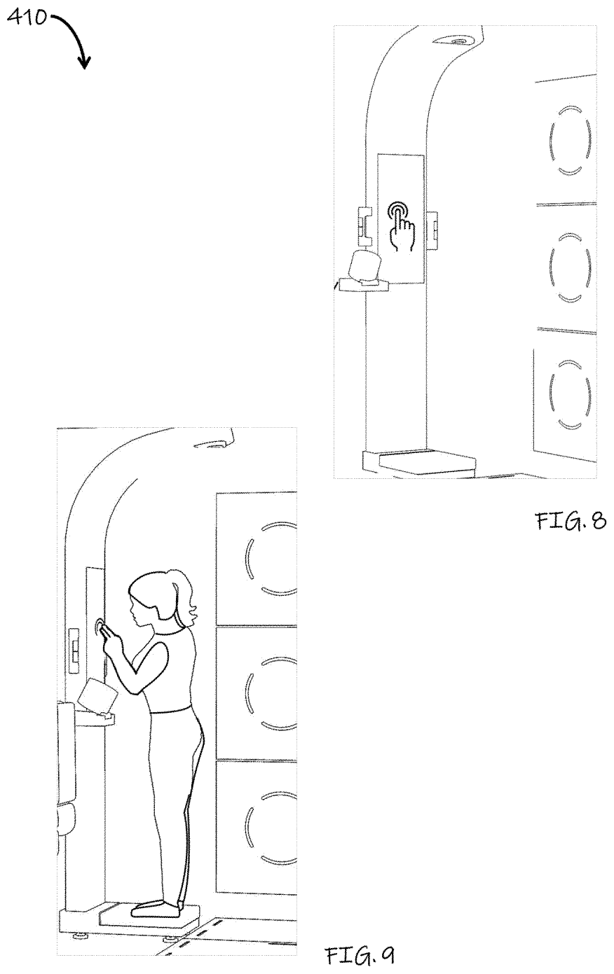

### DETAILED DESCRIPTION OF THE INVENTION

The following detailed description refers to the accompanying drawings, which illustrate specific embodiments. Other embodiments having different structures and operation do not depart from the scope of the present disclosure.

### LIST OF COMPONENT NUMERALS

100—Attributes of the invention as assembled in a custom mobile box truck/van.

110—Entry and access to vehicles.

150—Attributes of the invention as assembled in an expandable towed trailer.

170—Attributes of the invention as assembled in a floating watercraft.

200—Reactive floor and wall Surface Module.

220—Addressable LED array.

225—Pressure sensor array.

300—Mirror embedded leaderboard and streaming video instruction display Module.

410—Health Assessment Module.

420—Retractable Treatment and Therapy Couch Module.

500—Immersive Video Exergaming Module.

510—Tactile projection surface.

520—Video Projector with spatial sensor.

530—Interactive exergaming content.

550—Immersive Display Controller / Sensor.

600—Functional training aspects of the invention.

700—Logical and hierarchical technical system.

705—Main controller

730—Video display controller

740—Health Sensor Data Aggregator.

750—GPS and Vehicle Management System.

780—Smartphone/Tablet App.

770—Smartwatch App—body worn alarm sensor.

790—Cloud Management Platform.

The present invention relates to representative embodiments of mobile vehicles (100, 150, 170), without limitation to their form, with the primary objective of crafting them to serve as a mobile interactive health and fitness reactive and immersive activity space, crafted primarily as a mobile physical space (100, 150, 170) capable of being transported for mobility and abbreviated with a reactive flexible wall and floor surface module (200), a mirror embedded leader board and streaming video instruction display module (300) a health assessment module (410), a retractable treatment and therapy couch module (420) and an immersive video exergaming module (500).

The physical space is embodied with a plurality of modules, sensors, mood lighting and audio-visual cues that provide convenience, monitoring and mood enhancement. Professionals and users are provided a method of operating it using a smartphone/tablet app (780), with data exchange for user interaction with smart watches (770) and data exchange with a connected cloud platform (790).

The mobile vehicle (100, 150, 170) presents an open-plan and unobstructed machine-less activity space for conducting functional exercises (600) and is supported using suspension and resistance straps from the wall and ceiling beams to support functional strength and resistance exercises.

The invention is crafted by embodying a plurality of interactive sensory units which include, but are not limited to, sensors (not shown) that are capable of reading motion, pressure, weight, height, body measurements and bio-metric measurement that provide heart rate, blood pressure, blood oxygen level, electro-cardiography, electro-myography, lung capacity, blood glucose levels, to name a few, listed here for explanatory purposes only and without limitation to additional health sensors that are connected and integrated to the health sensor data aggregator (740).

The reactive wall and floor functional activity unit (200) has an addressable array of interactive light emitting diodes (220) and pressure sensors (225) encased inside a translucent top surface (230) and a rigid protective layer of flexible material (215) and a bottom layer of soft impact absorbing material (210).

The reactive floor and wall space (200) comprise of a translucent exercise surface that, in its current aspect, but not limited to this specific aspect in form and dimension, is fixed flat to the wall and floor using a square disposition and interlocking hooks to securely and strongly, interlock with each other.

All materials are impermeable materials that are dust and water resistant, easy to clean and disinfect with off the shelf disinfectants as well as known anti-bacterial and anti-viral ultra-violet frequency radiation and any other disinfectant means to ensure users can self-manage the hygiene under their personal supervision.

The mirror embedded leader board and streaming video instruction display module (300) is comprised to a two-way acrylic mirror panel (310) the size and thickness of which is determined by the wall sections where the module is deployed. The leader board and video instruction display (350) aspect of the module is comprised of a single or a plurality of high-quality video display system, the size and plurality of which is determined by the wall sections where the module is deployed. This two-part combined modular embodiment provides space-saving and functional benefit to users for visibly experiencing their own reflections while performing the exercises as well as for viewing instruction guided live or video content to facilitate such exercise. The leader board functionality shows connected users' performance and bio-metric information in a live, interactive information display, such as heart rates, speed, scores and other programmable information to enhance the quality of experience for the user.

The modular components of any embodiment of the invention may be physically, functionally, and logically implemented in any suitable way based on dimensional, occupant capacity, functionality and health and safety compliance considerations. The modules are integrated by the technical system into physical, logical and application layers (700A).

As one skilled in the art would realize that various other embodiments, shapes and sizes could be created with adaptations and added functionalities into vehicles that may be generally described to include trailers, wagons, motor vehicles (trucks, buses), railed vehicles (trains, trams), watercraft (ships, boats), amphibious vehicles (screw-propelled vehicle, hovercraft and towed barges), airplanes and spacecraft.

### How The Components Connect and Interact

The main controller (705) is embodied with a redundant high performance CPU and communication sub-systems equipped with wireless connectivity (706) which is equipped, but not limited to, using wired and wireless (WIFI, BLE, RF, IR communication technologies for interaction with sensors and equipment embodied with the mobile activity space (100, 150, 170), as well as connecting smartphones/tablet and smartwatch devices (780, 770)and a secure cloud platform (790) using, but not limited to, WIFI, 4G/5G GSM/LTE and mobile satellite connectivity.

The reactive wall and floor surface module (200), provides pressure sensing inputs to the controller via the pressure sensor array (225) and the visual cues and status indications during reactive functional training use are provided by the controller to the addressable LED array (220) using RGB color attributes. Together with the controller (705) the pressure sensors (225) and the LED array (220), users and professionals can operate reaction training aspect of the invention via their smartphone/tablet app (780) and receive guided information and haptic/visual/audible cues via personal body worn smartwatch app(770). The mirror embedded leaderboard and streaming video instruction display module (350) works in conjunction with the main controller (705), to provide connected streaming video and leaderboard visual score display related to the selected exercise program being executed by the professional/users within the activity space based on user selection of the program list from the smartphone/tablet app (780) in conjunction with the libraries stored in the connected cloud (790). High quality ambient surround sound audio is experienced via and embedded audio system in the activity space (not shown).

Similar to the video display (350) the immersive video exergaming module (500) provides high intensity and game-based exercise content, based on user selection of the program list from the smartphone/tablet app (780), in conjunction with the libraries stored in the connected cloud (790) that are projection displayed via the projector (520) and the tactile projection screen surface (510). Response and scoring data is processed via the immersive spatial sensor (550) and made visible on the screen via the interactive immersive content (530). The projection system is described here as one of the ways in which the immersive video exergaming module (500) will be created and specified here for explanatory purposes only and without limitation to use of other interactive display technologies and as one skilled in the art would realize that various other embodiments of interactive displays could be used with adaptations and added functionalities to fulfill the same purpose of providing an immersive video exergaming module.

The health assessment module (410) embodied with a plurality of health and bio-metric sensors provides individual sensor readings to the health sensor data aggregator (740) which provides it to the main controller (705) in conjunction with touch screen user inputs from the video display controller (730).

The physical mobile space (100, 150, 170) is embodied with a plurality of modules, sensors, mood lighting and audio-visual cues that provide convenience, monitoring and mood enhancement. Professionals and users are provided a method of operating it using a smartphone/tablet app (780), with data exchange for user interaction with smart watches (770) and data exchange with a connected cloud platform (790).

To support the performance of functional training, the physical mobile space (100, 150, 170) is embodied with ceiling beams (120) internal and external hooks (130), suspension systems (650) as shown in FIG. 17. These supports are weighted structurally to support a wide variety of body weights with tolerance to ensure safety and comfort. The location and plurality of these anchors, hooks and beams is representative in the drawings and actual layout, plurality and type will be determined based on the physical dimensional space and health and safety compliance considerations.

Location and operational tracking, summon exchanges, and vehicle management functions are embedded within the GPS/Vehicle Management device (750) that interacts with the secure cloud platform (790) to provide logs, service information and tracking data access for supporting assisted and totally autonomous functionality.

The main controller (705) has local data storage for securely and rapidly responding to the application, functional and media needs of the vehicle with minimal latency. Updates to the system are periodic, as needed, using over-the-air autonomous update mechanisms pushed via the secure cloud platform (790).

### Operation of the Invention

The present invention relates to representative embodiments of mobile vehicles (100, 150, 170), without limitation to their form, with the primary objective of crafting them to serve as a mobile interactive health and fitness reactive and immersive activity space, crafted primarily as a mobile physical space (100, 150, 170) capable of being transported for mobility and abbreviated with a reactive flexible wall and floor surface module (200), a mirror embedded leaderboard and streaming video instruction display module (300) a health assessment module (410), a retractable treatment and therapy couch module (420) and an immersive video exergaming module (500). The physical space is embodied with a plurality of modules, sensors, mood lighting and audio-visual cues that provide convenience, monitoring and mood enhancement. Professionals and users are provided a method of operating it using a smartphone/tablet app (780), with data exchange for user interaction with smart watches (770) and data exchange with a connected cloud platform (790).

The mobile vehicle (100, 150, 170) presents an open-plan and unobstructed machine-less activity space for conducting functional exercises (600) and is supported using suspension and resistance straps (650) from the wall hooks (130) and ceiling beams (120) to support functional strength and resistance exercises as demonstrated in FIG. 17.

The invention is meant to be used by two user types. Professional users who are primarily qualified and trained in the technical and functional operations of the invention and Functional users who use the invention for health assessment, physical and mental fitness training and treatment and therapy for physiology related ailments. The invention can cater to people of all ages using a library of activities and treatments to address people in all walks of their lives. User access is provided via the Smartphone/tablet app (780) which requires authenticated login via the connected secure cloud platform (790). The smartphone/tablet app (780) can distinguish the type of user and provides the user the user interface authorized for the user profile.

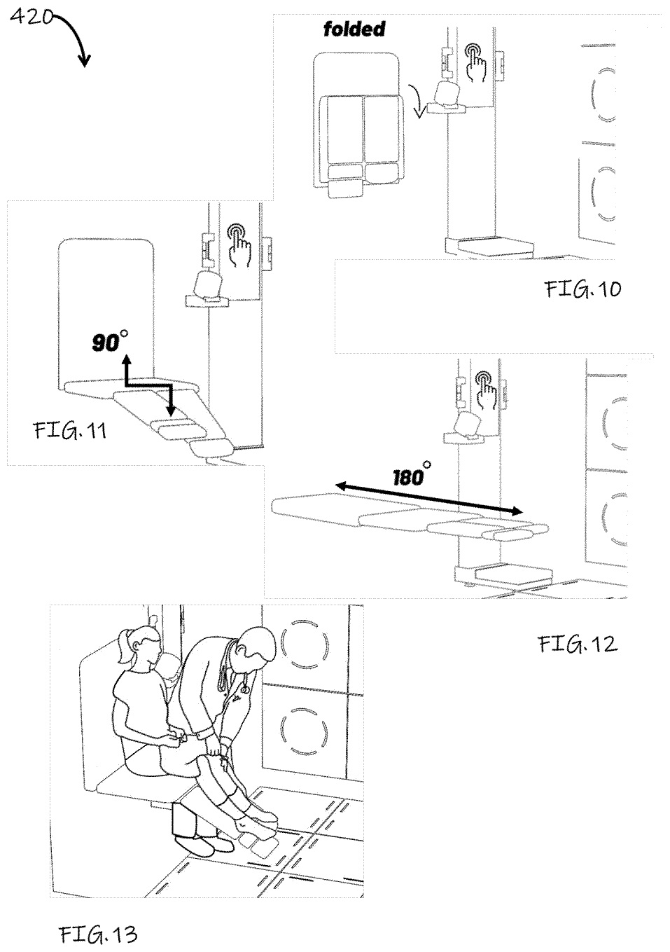

The health assessment module (410) embodied with a plurality of health and bio-metric sensors provides individual sensor readings to the health sensor data aggregator (740) which provides it to the main controller (705) in conjunction with touch screen user inputs from the video display controller (730) as depicted in FIG. 9. By standing on the floor platen of the health assessment module (410), the user is able to autonomously measure and record personal bio-metric information presented as bio-informatics such as body height and weight, body dimensions such as waist, shoulders, arms, legs, BMI, BMR, Body water, body fat, blood pressure, blood sugar, blood oxygen, body temperature, to name a few without limiting the scope of bio-analytics information protocols and processing which is an evolving science. The bioinformatics is presented to the user based on health metrics algorithms that are programmatically calculated in the secure cloud platform (790) and presented graphically to the user in the privacy of the personal smartphone app (780).

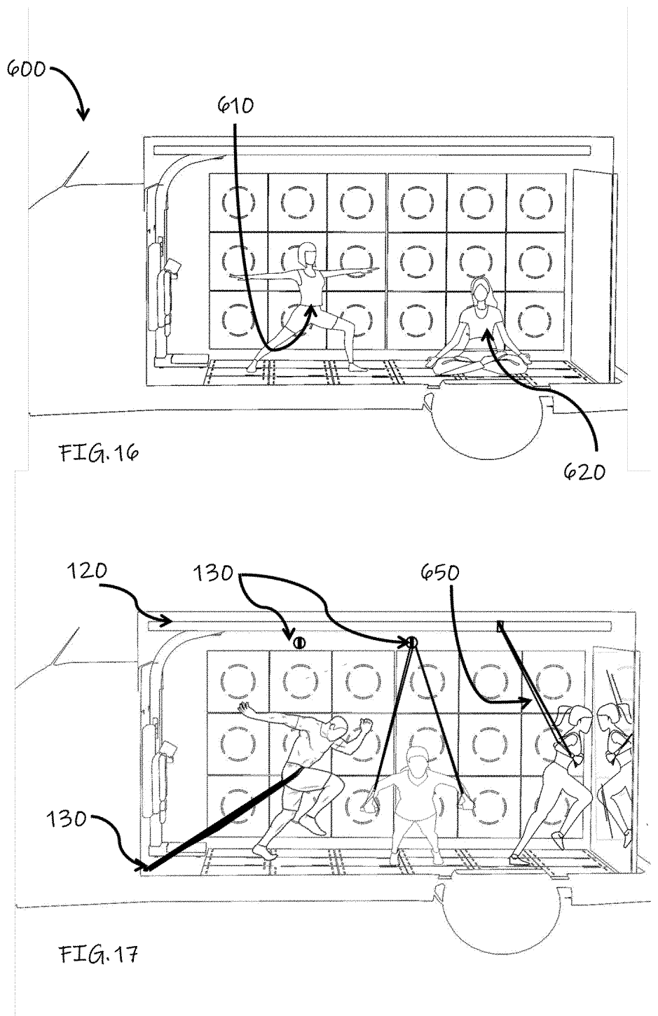

A physical health examination (FIG. 13) can be performed by the professional user using the treatment and therapy couch (420) based on the bio-analytics results and physical examination. The professional user can record notes and pictures and videos of the examination. Based on privacy and safety compliances required, this area may be screened using ceiling mounted opaque curtain arrays (not shown), while being used.

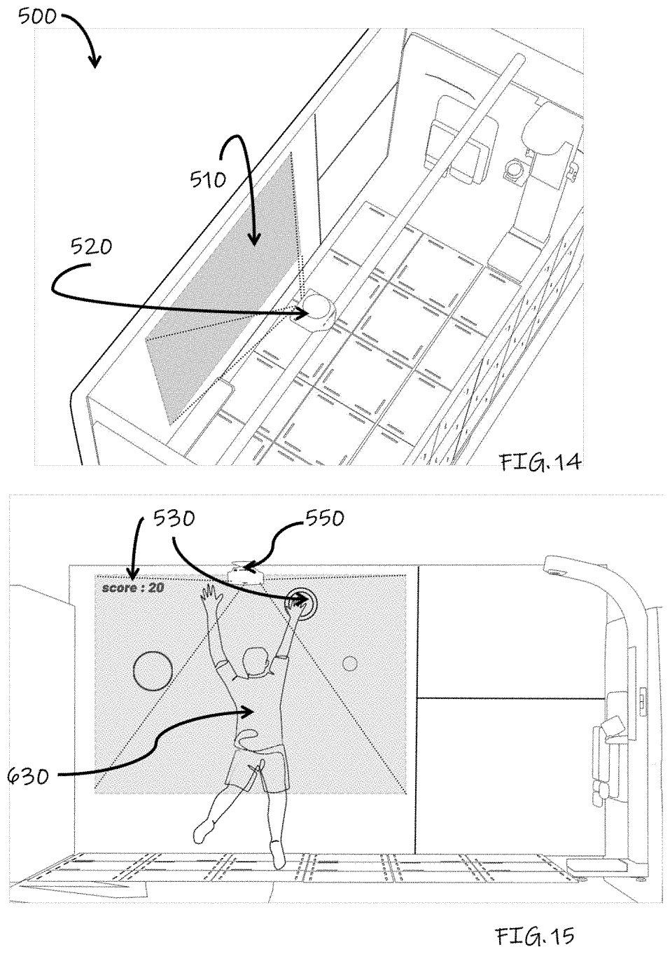

Based on the qualified health bio-analytics and physical examination the professional user can identify a bespoke physical and mental health fitness regimen and therapies for the user based on pre-programmed protocols and exercise libraries available via the secure cloud platform (790). Physical therapy and treatments can be provided to functional users on the retractable treatment and therapy couch (420) which adjusts to a sitting position (FIG. 11) as well as a supine position (FIG. 12).

Progressive health data is privately and secured stored in the user profile management in the cloud platform (790) and provides periodic tracking information to the professional and personal user for evidencing the results and augmenting the benefits of the using guided consultation.

Functional operation of the invention is normally conducted while the vehicle is parked and immobile for safety considerations and in compliance to vehicle safety operational compliances.

The invention's novelty is that it provides a mobile health and fitness interactive and immersive activity space that delivers bespoke functional training and therapy as an efficient alternative to traditional brick-and-mortar gyms and mobile fitness units that are laden with machines and weight training equipment.

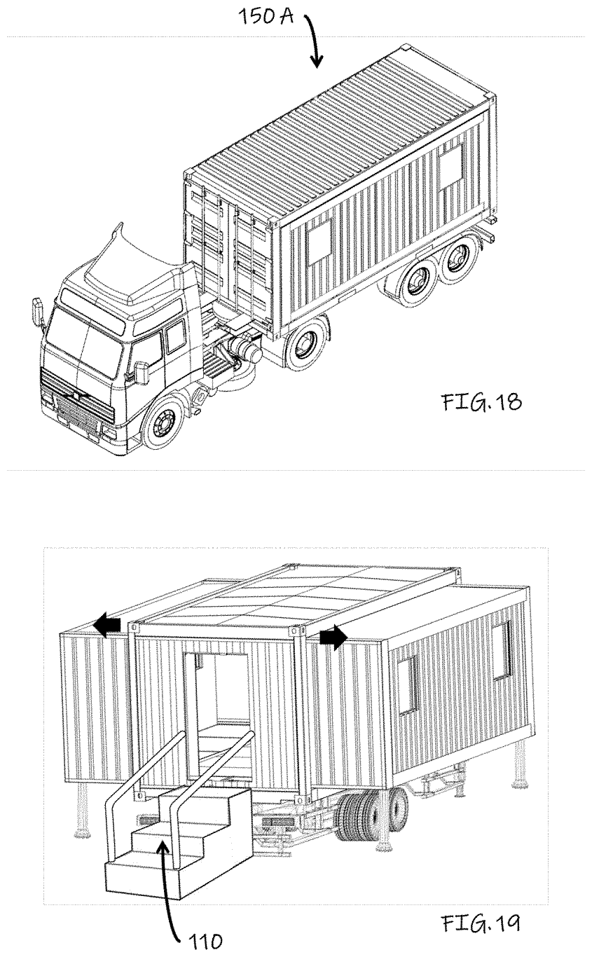

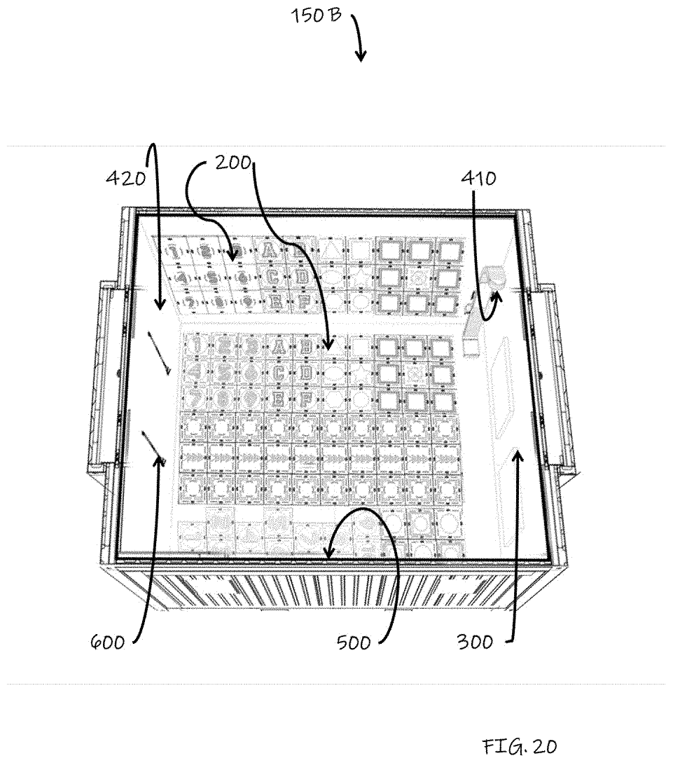

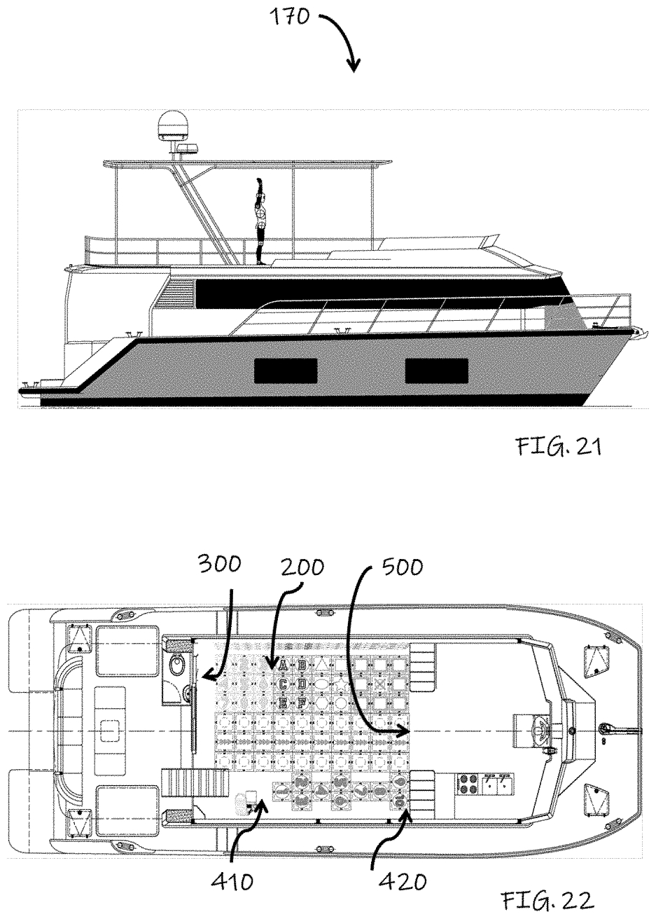

The revelation details a modular mobile interactive and immersive health and fitness space using reactive floor and walls for high intensity functional training, with the objective of reducing the fitness activity time by as much as eighty percent, while providing similar calorie burn as compared to a traditional cardio machines and weights driven exercise training system. Depending on the fitness status of the individual functional user, a plurality of functional fitness exercises can be performed within the activity space (100, 150, 170) as depicted in FIG. 15, FIG. 16 and FIG. 17., catering to providing bespoke training including hand eye coordination and cognitive training to a wide variety of individuals from children to aged individuals.

### ALTERNATIVE EMBODIMENTS

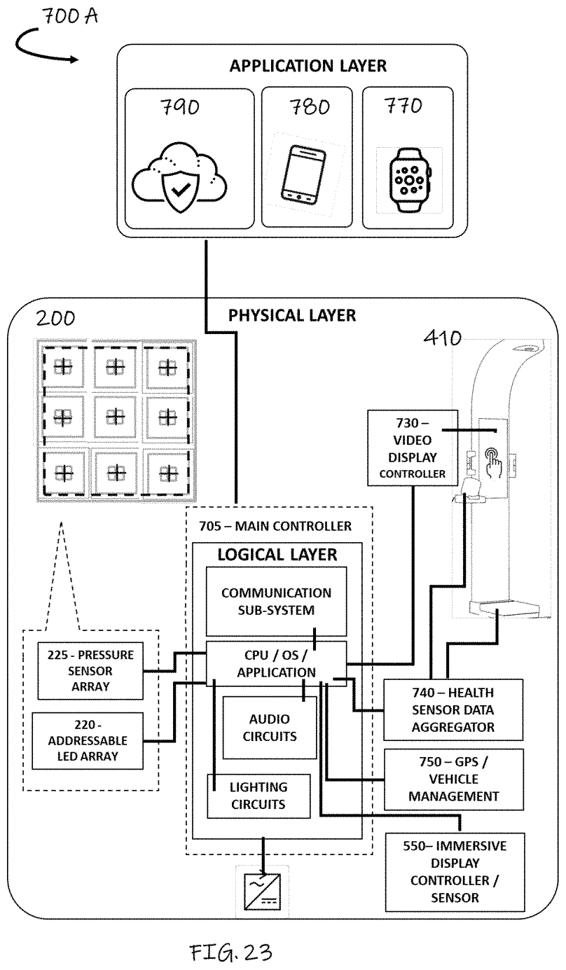

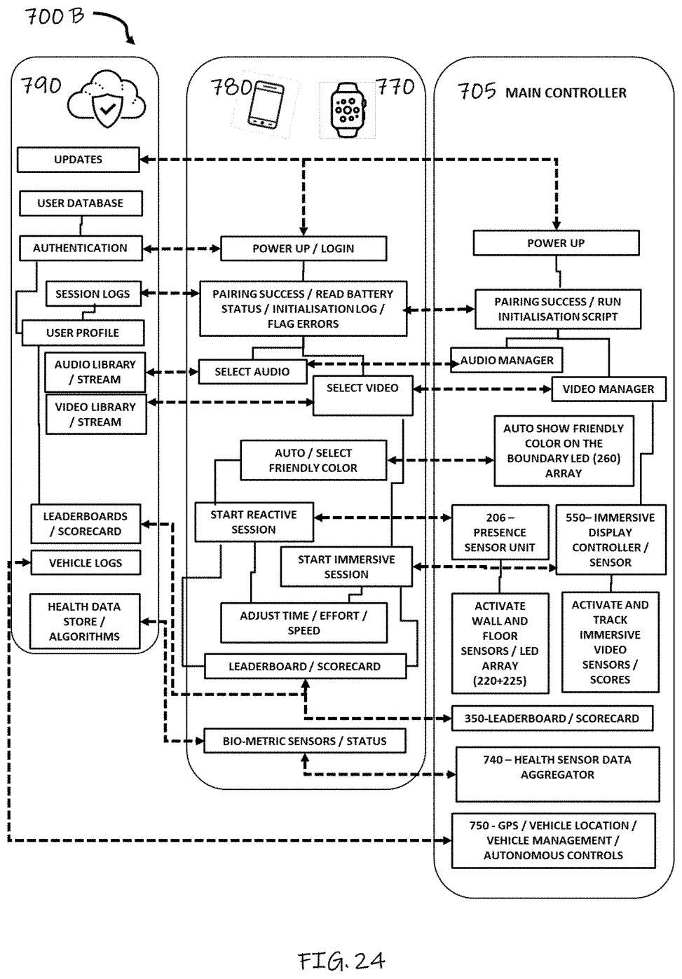

The invention can be implemented in several combinations based on the tiered architecture of the system (FIG. 23 and FIG. 24). The hardware, application software, firmware, or any combination of these can be adapted for use in an embodiment of the invention which may be physically, functionally and logically implemented in any suitable way. Indeed, the functionality may be implemented in a single unit, in a plurality of units or as part of a group of other functional units or may be physically and functionally distributed between different units and processors.

Although the present invention has been described in connection with the specified embodiments, it is not intended to be limited to the specific form set forth herein.

Additionally, although individual features may be defined, these may possibly be advantageously combined, and the inclusion in different parts of the invention revelation does not imply that a combination of features is not feasible and/or advantageous. In addition, singular references do not exclude a plurality. Thus, references to “a”, “an”, “first”, “second” etc.do not preclude a plurality. Furthermore, reference labels shall not be construed as limiting the scope of this invention.

Numerous modifications and adaptations of the system of the present invention will be apparent to those skilled in the art, and thus it is intended by the appended claims to cover all such modifications and adaptations which fall within the true spirit and scope of this invention.

What is claimed is:

1. A mobile health and fitness reactive immersive activity space which comprises:
a mobile physical space (100, 150, 170) capable of being transported for mobility and abbreviated with a reactive flexible wall and floor surface module (200),
a mirror embedded leader board and streaming video instruction display module (300) a health assessment module (410),
a retractable treatment and therapy couch module (420) and an immersive video exergaming module (500) to provide high intensity and game based exercise content, based on user selection of the program list from the smartphone/tablet app (780), in conjunction with the libraries stored in the connected cloud (790) that are projection displayed via the projector (520) and the tactile projection screen surface (510) and response and scoring data is processed via the immersive spatial sensor (550) and made visible on the screen via the interactive immersive content (530),
a plurality of interactive sensory units which include, but are not limited to, sensors (not shown) that are capable of reading motion, pressure, weight, height, body measurements and bio-metric measurement that provide heart rate, blood pressure, blood oxygen level, electro-cardiography, electro-myography, lung capacity, blood glucose levels, to name a few, listed here for explanatory purposes only and without limitation to additional sensors that are connected and integrated to the health sensor data aggregator (740),
reactive wall and floor functional activity unit (200) with an addressable array of interactive light emitting diodes (220) and pressure sensors (225) encased inside a translucent top surface (230) and a rigid protective layer of flexible material (215) and a bottom layer of soft impact absorbing material (210),
main controller (705) embodied with a redundant high-performance CPU and communication sub-systems equipped with wireless connectivity (706) which is equipped, but not limited to, using wired and wireless communication for interaction with sensors and equipment embodied with the mobile activity space (100, 150, 170), as well as connecting smartphones/tablet and smartwatch devices (780, 770) and a secure cloud platform (790)

2. The mobile vehicle, as claimed in claim 1, wherein the said vehicle (100, 150, 170) presents an open-plan and unobstructed machine-less activity space for conducting functional exercises (600) and is supported using suspension and resistance straps from the wall and ceiling beams to support functional strength and resistance exercises.

3. The mobile vehicle, as claimed in claim 1, wherein the said reactive floor and wall space (200) comprise of a translucent exercise surface that, in its current aspect, but not limited to this specific aspect in form and dimension, is fixed flat to the wall and floor using a square disposition and interlocking hooks to securely and strongly interlock with each other.

4. The reactive floor and wall space (200), as claimed in claim 3, wherein the reactive wall and floor surface module (200), provides pressure sensing inputs to the controller via the pressure sensor array (225) and the visual cues and status indications during reactive functional training use are provided by the controller to the addressable LED array (220) using RGB color attributes.

5. The reactive floor and wall space (200), as claimed in claim 4, wherein the controller (705) the pressure sensors (225) and the LED array (220), operates reaction training aspect via their smartphone/tablet app (780) and receive guided information and haptic/visual/audible cues via personal body worn smartwatch app (770).

6. The mobile vehicle, as claimed in claim 1, wherein the health assessment module (410) is embodied with a plurality of health and bio-metric sensors provides individual sensor readings to the health sensor data aggregator (740) which provides it to the main controller (705) in conjunction with touch screen user inputs from the video display controller (730)

7. The health assessment module (410), as claimed in claim 6, wherein based on the qualified health bio-analytics and physical examination the professional user can identify a bespoke physical and mental health fitness regimen and therapies for the user based on pre-programmed protocols and exercise libraries available via the secure cloud platform (790).

8. The health assessment module (410), as claimed in claim 7, wherein physical therapy and other treatments can be provided to functional users on the retractable treatment and therapy couch (420) which adjusts to a sitting position as well as a supine position within pressurized as well as de-pressurized hyperbaric environments, based on privacy and safety compliances required, using opaque curtain arrays.
